(19) United States
(12) Patent Application Publication
(10) Pub. No.: US 2013/0052680 A1
(43) Pub. Date: Feb. 28, 2013

(54) APPARATUS AND METHOD FOR EVALUATION OF ANTIMICROBIAL AIR FILTER EFFICIENCY

(71) Applicants: Gwi Nam BAE, Seoul (KR); Seung Jae LEE, Jeju-do (KR); Jae Hee JUNG, Chungcheongbuk-do (KR); Seung Bok LEE, Seoul (KR); Gi Byoung HWANG, Gyeongsangbuk-do (KR)

(72) Inventors: Gwi Nam BAE, Seoul (KR); Seung Jae LEE, Jeju-do (KR); Jae Hee JUNG, Chungcheongbuk-do (KR); Seung Bok LEE, Seoul (KR); Gi Byoung HWANG, Gyeongsangbuk-do (KR)

(73) Assignee: Korea Institute of Science and Technology, Seoul (KR)

(21) Appl. No.: 13/437,115

(22) Filed: Apr. 2, 2012

(30) Foreign Application Priority Data
Aug. 25, 2011 (KR) 10-2011-0085303

(51) Int. Cl. G01N 21/64; C12M 1/34

(52) U.S. Cl. 435/32; 435/288.7

(57) ABSTRACT

Disclosed are an apparatus and a method for evaluating the efficiency of an antimicrobial air filter, which allow precise determination of the efficiency of an antimicrobial air filter by dividing microorganisms deposited on the antimicrobial air filter into living microorganisms and dead microorganisms by use of two types of fluorescent dyes, and by analyzing the microorganisms quantitatively.

### CROSS-REFERENCE TO RELATED APPLICATION

This application claims priority to Korean Patent Application No. 10-2011-0085303, filed on Aug. 25, 2011, and all the benefits accruing therefrom under 35 U.S.C. §119, the contents of which in its entirety are herein incorporated by reference.

### BACKGROUND

1. Field

The present disclosure relates to an apparatus and a method for evaluating an efficiency of an antimicrobial air filter.

2. Description of the Related Art

In general, indoor air may include various kinds of microorganisms, such as bacteria, mold or virus suspending therein. Such indoor suspending microorganisms may cause aerial infection and environmental diseases, thereby adversely affecting human health. Such indoor suspending microorganisms may be primarily filtered off through an air filter which is capable of removing dust. However, since the microorganisms are survivable, they may proliferate on the surface of a filter media so as to emit microbial volatile organic compounds harmful to the human body or they may be reintroduced into the indoor environment.

Recently, to address the above-mentioned problems, there have been suggested methods for preventing proliferation of microorganisms by applying an inorganic antimicrobial agent, such as silver, copper, gold, carbon nanotube, or TiO₂, or an organic antimicrobial agent, such as catechin, chitosan, phytoncide, Hosta capitata extract, sophora root extract, gingko leaf extract, herb extract, pine leaf extract or maple leaf extract to the surface of a filter media. For example, there has been known a filter media containing silver nanoparticles.

Meanwhile, it may be required to evaluate a performance of the filter media to which the antimicrobial agent is applied. Typical methods for testing an efficiency of the filter media treated with the antimicrobial agent may include liquid extraction test and agar diffusion test. The liquid extraction test may include extracting microorganisms collected at the filter media, swabbing the microorganisms onto a culture plate and determining a grown microorganism colony. The agar diffusion method may include swabbing microorganisms onto a culture plate, attaching the filter media thereto, and determining a growth prohibition radius.

However, according to the inventors' study, such liquid extraction test and agar diffusion test according to the related art may require excessive intermediate treatment operations to determine antimicrobial efficiency. In addition, in case of the diffusion method, efficiency is measured in an indirect manner because the method determines the growth prohibition radius. Therefore, the results of antimicrobial efficiency may significantly depend on a particular type of the filter media and a particular antimicrobial treatment of the filter media. Further, during the evaluation of antimicrobial efficiency, the antimicrobial agent applied to the filter media may be extracted concurrently or subjected to physical stress.

### SUMMARY

The present disclosure is directed to provide an apparatus and a method for evaluating an efficiency of an antimicrobial air filter, which may allow precise determination of the efficiency of the antimicrobial air filter by dividing microorganisms deposited on the antimicrobial air filter into living microorganisms and dead microorganisms by using two types of fluorescent dyes, and by analyzing the microorganisms quantitatively through a fluorescence analysis system.

In exemplary embodiments, an apparatus for evaluating the efficiency of an antimicrobial air filter includes: a microorganism-supplying device supplying a microorganism-containing solution by spraying the microorganism-containing solution in a gas phase; a drying device drying the microorganism-containing solution sprayed in a gas phase to dry a solvent in the solution; an antimicrobial air filter chamber provided with an antimicrobial air filter and providing a space in which the microorganisms supplied from the drying device are attached to the antimicrobial air filter; a fluorescent dyeing device for the antimicrobial air filter carrying out fluorescent dyeing of the antimicrobial air filter by using a fluorescent dye containing a combination of a first fluorescent dye capable of fluorescent dyeing of living microorganisms and a second fluorescent dye capable of fluorescent dyeing of dead microorganisms; and a fluorescence analysis device measuring the fluorescence emitted from the antimicrobial air filter fluorescent-dyed by the fluorescent dyeing device to perform quantitative analysis of the living microorganisms and dead microorganisms.

The first fluorescent dye and the second fluorescent dye may emit fluorescence which differs from each other in wavelength. The first fluorescent dye may be Syto9 and the second fluorescent dye may be propidium iodide.

The microorganism-containing solution sprayed in a gas phase and the microorganisms in the drying device may be transferred by compressed air, and the compressed air may be generated by an air compressor.

The fluorescent dyeing device for the antimicrobial air filter may include a fluorescent dye supplying device and a substrate. The antimicrobial air filter having the microorganisms attached thereto are mounted to the substrate, and the fluorescent dyes are sprayed onto the antimicrobial air filter through the fluorescent dye supplying device, so that the antimicrobial air filter is dyed with the fluorescent dye.

The fluorescence analysis device may include a fluorescence filter block and a fluorescence microscope. The fluorescence filter block may serve to filter the specific fluorescence emitted from each of the fluorescent-dyed living microorganisms and dead microorganisms. The fluorescence microscope may recognize the shapes of microorganisms and the fluorescence emitted from microorganisms, and thus may determine number of the living microorganisms and that of the dead microorganisms.

In another exemplary embodiments, a method for evaluating the efficiency of an antimicrobial air filter may include supplying a microorganism-containing solution by spraying the microorganism-containing solution in a gas phase; drying the microorganism-containing solution sprayed in a gas phase to dry a solvent in the solution; attaching microorganisms dried from the solution to an antimicrobial air filter; and carrying out fluorescent dyeing of the antimicrobial air filter by using a fluorescence dye containing a combination of a first fluorescent dye capable of fluorescent dyeing of living microorganisms and a second fluorescent dye capable of fluorescent dyeing of dead microorganisms; and measuring the fluorescence emitted from the antimicrobial air filter dyed with the first fluorescent dye and the second fluorescent dye to determine number of living microorganisms and that of dead microorganisms.

For example, the method for evaluating the efficiency of the antimicrobial air filter may use the apparatus for evaluating the efficiency of the antimicrobial air filter which may include the microorganism-supplying device, the drying device, the antimicrobial air filter chamber, the fluorescent dyeing device for the antimicrobial air filter, and the fluorescence analysis device.

More particularly, the method for evaluating the efficiency of the antimicrobial air filter may include spraying a microorganism-containing solution in a gas phase by the microorganism-supplying device; drying the microorganism-containing solution sprayed in a gas phase by the drying device so that the microorganisms are supplied to the antimicrobial air filter chamber; attaching the microorganisms supplied from the drying device to the antimicrobial air filter provided in the antimicrobial air filter chamber; mounting the antimicrobial air filter having the microorganisms attached thereto to the substrate of the fluorescent dyeing device for the antimicrobial air filter, and carrying out fluorescent dyeing of the antimicrobial air filter by using a fluorescence dye containing a combination of a first fluorescent dye capable of fluorescent dyeing of living microorganisms and a second fluorescent dye capable of fluorescent dyeing of dead microorganisms; and measuring the fluorescence emitted from the antimicrobial air filter dyed with the first fluorescent dye and the second fluorescent dye to determine the number of living microorganisms and that of dead microorganisms.

According to the apparatus and method for evaluating the efficiency of the antimicrobial air filter disclosed herein, it is possible to carry out a realistic simulation of the collection of microorganisms on an antimicrobial air filter during purification of indoor air and to uniformly distribute microorganisms on the antimicrobial air filter. As a result, it is possible to evaluate the efficiency of the antimicrobial air filter more objectively.

As mentioned above, the liquid extraction test and the agar diffusion test may have disadvantages in that they require complicated intermediate treatment procedure and they are indirect measuring. By contrast, the apparatus and method disclosed herein may determine the microorganisms collected by the antimicrobial air filter in a direct manner without a need for a separate operation of extracting the microorganisms. Therefore, the apparatus and method disclosed herein may evaluate the efficiency of the antimicrobial air filter in a simple manner with high accuracy.

### BRIEF DESCRIPTION OF THE DRAWINGS

The above and other aspects, features and advantages of the disclosed exemplary embodiments will be more apparent from the following detailed description taken in conjunction with the accompanying drawings in which:

FIG. 1 is a schematic view illustrating the apparatus for evaluating the efficiency of an antimicrobial air filter according to an example;

FIG. 2 is a schematic view illustrating the microorganism-supplying device, the drying device and the antimicrobial air filter chamber according to an example;

FIG. 3 is a schematic view illustrating fluorescent dyeing and fluorescence analysis using the fluorescent dyeing device for an antimicrobial air filter and the fluorescence microscope according to an example;

FIG. 4 is a graph showing the size distribution of the microorganisms generated by the apparatus for evaluating the efficiency of an antimicrobial air filter according to an example;

FIG. 5 is a fluorescence microscope image of the microorganisms attached to the antimicrobial air filter by the apparatus for evaluating the efficiency of an antimicrobial air filter according to an example;

FIG. 6 is a fluorescence microscope image showing the fluorescence response of the microorganisms collected on a conventional membrane filter and on an antimicrobial air filter dependent on time according to the same example as FIG. 5; and

FIG. 7 is a graph showing the results of the survival rate of microorganisms obtained by measuring the fluorescence intensities in a test according to the same example as FIG. 6.

### DETAILED DESCRIPTION

Exemplary embodiments now will be described more fully hereinafter with reference to the accompanying drawings, in which exemplary embodiments are shown. The present disclosure may, however, be embodied in many different forms and should not be construed as limited to the exemplary embodiments set forth therein. Rather, these exemplary embodiments are provided so that the present disclosure will be thorough and complete, and will fully convey the scope of the present disclosure to those skilled in the art. In the description, details of well-known features and techniques may be omitted to avoid unnecessarily obscuring the presented embodiments.

The terminology used herein is for the purpose of describing particular embodiments only and is not intended to be limiting of the present disclosure. As used herein, the singular fiat ns “a”, “an” and “the” are intended to include the plural forms as well, unless the context clearly indicates otherwise. Furthermore, the use of the terms a, an, etc. does not denote a limitation of quantity, but rather denotes the presence of at least one of the referenced item. The use of the terms “first”, “second”, and the like does not imply any particular order, but they are included to identify individual elements. Moreover, the use of the terms first, second, etc. does not denote any order or importance, but rather the terms first, second, etc. are used to distinguish one element from another. It will be further understood that the terms “comprises” and/or “comprising”, or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

In the drawings, like reference numerals denote like elements. The shape, size and regions, and the like, of the drawing may be exaggerated for clarity.

In the embodiments, the efficiency of a filter media to which an antimicrobial agent is applied (i.e., antimicrobial air filter) is evaluated quantitatively. For this, microorganisms are attached to an antimicrobial air filter. After a certain period of time, then living microorganisms and dead microorganisms are dyed with different kinds of fluorescent dyes, and the microorganisms in each case are analyzed quantitatively to determine the efficiency of the antimicrobial air filter.

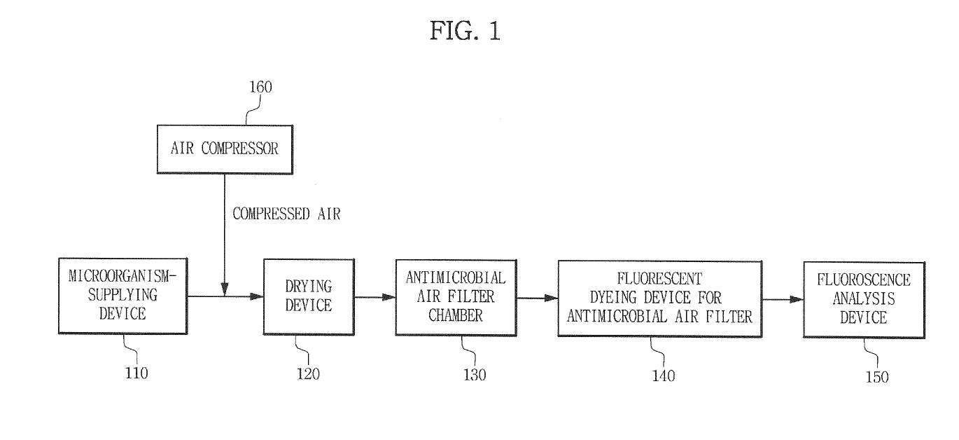

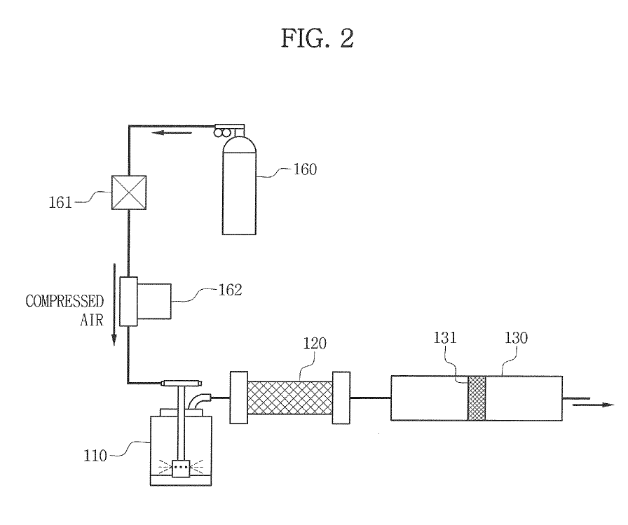

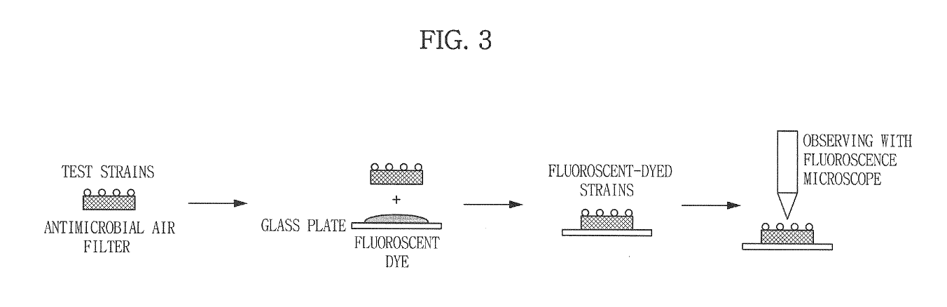

FIG. 1 is a schematic view illustrating the apparatus for evaluating the efficiency of an antimicrobial air filter according to an example. FIG. 2 is a schematic view illustrating the microorganism-supplying device, the drying device and the antimicrobial air filter chamber according to an example. FIG. 3 is a schematic view illustrating fluorescent dyeing and fluorescence analysis using the fluorescent dyeing device for an antimicrobial air filter and the fluorescence microscope according to an example.

Referring to FIGS. 1, 2 and 3, the apparatus for evaluating the efficiency of an antimicrobial air filter according to an example may include a microorganism-supplying device 110, a drying device 120, an antimicrobial air filter chamber 130, a fluorescent dyeing device 140 for the antimicrobial air filter, and a fluorescence analysis device 150.

The microorganism-supplying device 110 may supply a microorganism-containing solution by spraying the microorganism-containing solution in a gas phase. The microorganism-containing solution sprayed in a gas phase may be introduced to the drying device 120 by compressed air. The compressed air may be generated by an air compressor 160, and the flow of compressed air may be controlled by an air flow meter 162. A separate filter 161 may be further provided on the flow path of the compressed air in order to remove impurities contained in the compressed air. Herein, the microorganisms refer to strains with which the antimicrobial air filter is contaminated. The amount of supplied microorganisms may be determined by controlling the flow of compressed air, the concentration of microorganism-containing solution, or the like.

The drying device 120 may dry the microorganism-containing solution generated and supplied by the microorganism-supplying device 110. The drying device 120 may allow the solvent component to evaporate, and thus only the microorganisms, i.e., strains are supplied to the antimicrobial air filter chamber 130 which may be provided at the rear end of the drying device 120. The strains from which the solvent component is removed may be transferred to the antimicrobial air filter chamber 130 with the aid of compressed air.

The antimicrobial air filter chamber 130 may be a chamber provided with an antimicrobial air filter 131 and provide a space in which microorganisms are able to be attached to the surface of the antimicrobial air filter. The microorganisms supplied from the drying device 120 may be attached to the antimicrobial air filter of the antimicrobial air filter chamber 130. The antimicrobial air filter refers to a filter media coated with an antimicrobial agent. Particular examples of the antimicrobial agent that may be used herein may include an inorganic antimicrobial agent, such as silver (Ag), titanium dioxide (TiO₂) or carbon nanotubes (CNT), or a natural organic antimicrobial agent, such as chitosan, phytoncide, Hosta capitata extract or sophora root extract. The amount of the microorganisms attached to the antimicrobial air filter may be determined by the amount of the microorganisms supplied by the microorganism-supplying device 110 and by the time during which the microorganisms are collected on the antimicrobial air filter.

The microorganisms attached to the antimicrobial air filter may be destructed by the antimicrobial agent of the antimicrobial air filter. The degree of destruction of microorganisms caused by the antimicrobial air filter (i.e., the efficiency of the antimicrobial air filter) may be evaluated by the fluorescent dyeing device 140 for the antimicrobial air filter and the fluorescence analysis device 150.

The fluorescent dyeing device 140 for the antimicrobial air filter may carry out fluorescent dyeing of the antimicrobial air filter by using fluorescent dyes. More particularly, the fluorescent dyeing device 140 for the antimicrobial air filter may include a fluorescent dye-supplying device and a substrate. The antimicrobial air filter having the microorganisms attached thereto in the antimicrobial air filter chamber 130 for a certain period of time may be mounted to the substrate, and fluorescent dyes may be sprayed to the antimicrobial air filter through the fluorescent dye-supplying device so that the antimicrobial air filter is dyed with the fluorescent dyes. In another embodiment, fluorescent dyes may be sprayed onto the substrate, and then the antimicrobial air filter may be dipped into the fluorescent dyes so as to carry out fluorescent dyeing of the antimicrobial air filter.

The fluorescent dyes supplied by the fluorescent dye-supplying device may have a combination of a first fluorescent dye and a second fluorescent dye. The first fluorescent dye may serve to carry out fluorescent dyeing of living microorganisms and the second fluorescent dye may serve to carry out fluorescent dyeing of dead microorganisms. The first fluorescent dye may be bound to the cell walls of the living microorganisms, thereby emitting green fluorescence at a wavelength of about 520 nm to about 550 nm. In addition, the second fluorescent dye may be bound to the nucleic acids of the dead microorganisms, thereby emitting red fluorescence at a wavelength of about 660 nm to about 680 nm. For example, Syto9 and propidium iodide may be used as the first fluorescent dye and the second fluorescent dye, respectively.

As described above, the living microorganisms and the dead microorganisms attached to the antimicrobial air filter may be dyed with the first fluorescent dye and the second fluorescent dye, respectively, so that the living microorganisms and the dead microorganisms emit fluorescence at a wavelength different from each other.

The fluorescence analysis device 150 may measure the fluorescence emitted from the antimicrobial air filter dyed with the fluorescent dyes by the fluorescent dyeing device 140 for the antimicrobial air filter, and carry out quantitative analysis of the living microorganisms and the dead microorganisms. More particularly, the fluorescence analysis device 150 may include a fluorescence filter block and a fluorescence microscope. The fluorescence filter block may serve to filter the specific fluorescence emitted from each of the living microorganisms and dead microorganisms dyed with the fluorescent dyes. The fluorescence microscope may serve to recognize the shapes of microorganisms and the fluorescence emitted from microorganisms. The fluorescence microscope may further include an image processing device. By using the image processing device, it is possible to determine the number of total microorganisms emitting fluorescence.

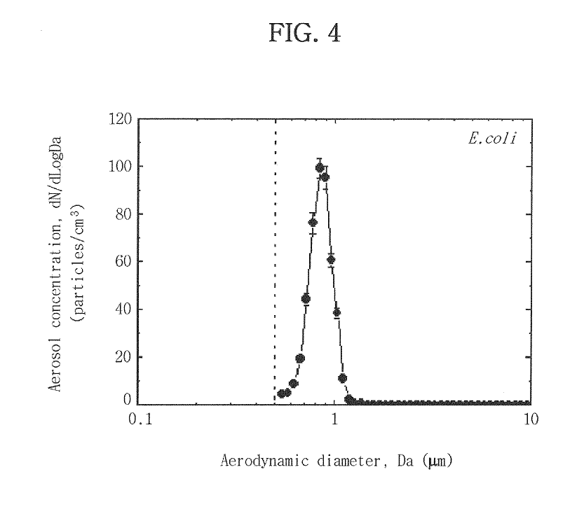

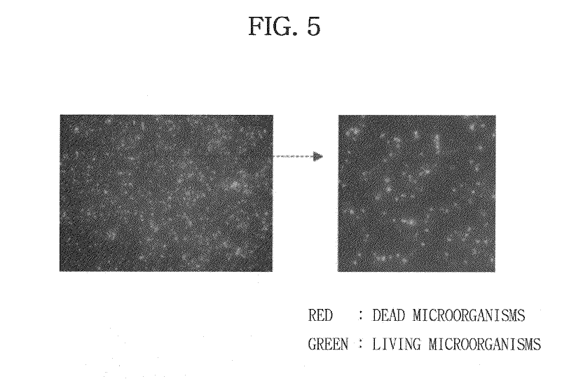

Hereinafter, the microorganisms (strains) and the antimicrobial air filter according to an example will be further explained. FIG. 4 is a graph showing the size distribution of the microorganisms generated by the apparatus for evaluating the efficiency of an antimicrobial air filter according to an example. FIG. 5 is a fluorescence microscope image of the microorganisms attached to the antimicrobial air filter by the apparatus for evaluating the efficiency of an antimicrobial air filter according to an example.

FIG. 4 shows the results of size distribution measured after the test strains (E. coli) are distributed into the air. The liquid sample containing E. coli has a concentration of about 1×10⁷ CFU/mL (colony forming unit/mL) and the compressed air has a flow rate of 1 L/min. In FIG. 4, X axis shows a diameter of microorganism and Y axis shows a concentration of microorganism as a function of size. As shown in FIG. 4, microorganisms having the highest concentration show a diameter of about 0.85 μm and are distributed with a narrow monodisperse size distribution.

FIG. 5 shows fluorescence of the microorganisms attached to the surface of the antimicrobial air filter and dyed with the fluorescent dyes, which was measured with a fluorescence microscope. It is possible to calculate the survival rate of the total microorganisms by counting microorganisms which are expressed by two different types of fluorescence, and thus to evaluate the efficiency of the antimicrobial air filter.

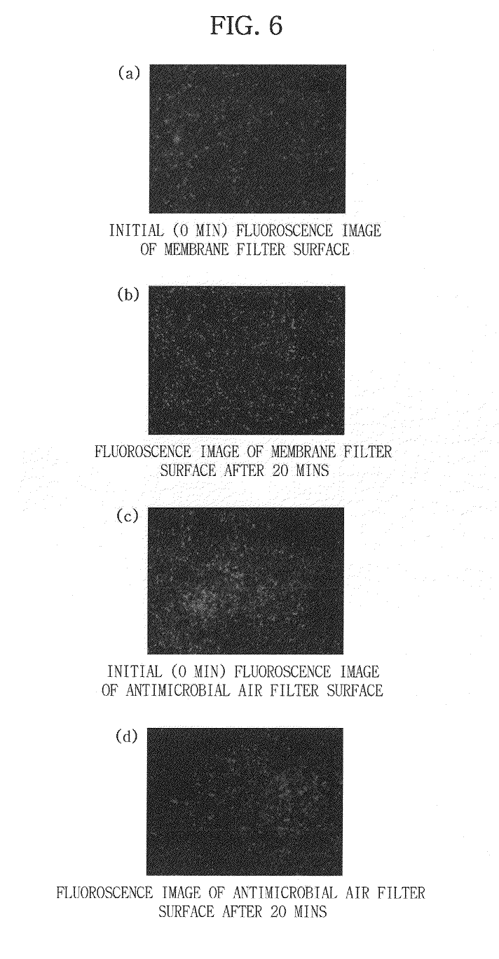

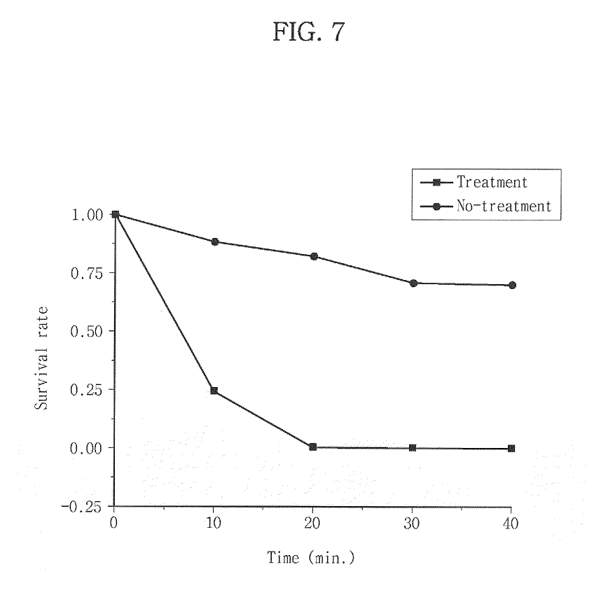

FIG. 6 is a fluorescence microscope image showing the fluorescence response of the microorganisms collected on a conventional membrane filter and on an antimicrobial air filter dependent on time according to the same example as FIG. 5. FIG. 7 is a graph showing the results of the survival rate of microorganisms obtained by measuring the fluorescence intensities in a test according to the same example as FIG. 6. As can be seen from FIG. 6 and FIG. 7, the apparatus for evaluating the efficiency of an antimicrobial air filter disclosed herein may enable determination of the efficiency of an antimicrobial air filter in a quasi-realtime manner.

In another exemplary embodiments, a method for evaluating the efficiency of an antimicrobial air filter may include supplying a microorganism-containing solution by spraying the microorganism-containing solution in a gas phase, drying the microorganism-containing solution sprayed in a gas phase to dry a solvent in the solution, attaching microorganisms dried from the solution to an antimicrobial air filter, and carrying out fluorescent dyeing of the antimicrobial air filter by using a fluorescence dye containing a combination of a first fluorescent dye capable of fluorescent dyeing of living microorganisms and a second fluorescent dye capable of fluorescent dyeing of dead microorganisms, and measuring the fluorescence emitted from the antimicrobial air filter dyed with the first fluorescent dye and the second fluorescent dye to determine number of living microorganisms and that of dead microorganisms.

For example, a method for evaluating the efficiency of an antimicrobial air filter may use the above-mentioned apparatus for evaluating the efficiency of an antimicrobial air filter which may include the microorganism-supplying device, the drying device, the antimicrobial air filter chamber, the fluorescent dyeing device for the antimicrobial air filter, and the fluorescence analysis device.

More particularly, the method for evaluating the efficiency of an antimicrobial air filter may include spraying a microorganism-containing solution in a gas phase by the microorganism-supplying device, drying the microorganism-containing solution sprayed in a gas phase by the drying device so that the microorganisms are supplied to the antimicrobial air filter chamber, attaching the microorganisms supplied from the drying device to the antimicrobial air filter provided in the antimicrobial air filter chamber, mounting the antimicrobial air filter having the microorganisms attached thereto to the substrate of the fluorescent dyeing device for the antimicrobial air filter, and carrying out fluorescent dyeing of the antimicrobial air filter by using a fluorescence dye containing a combination of a first fluorescent dye capable of fluorescent dyeing of living microorganisms and a second fluorescent dye capable of fluorescent dyeing of dead microorganisms, and measuring the fluorescence emitted from the antimicrobial air filter dyed with the first fluorescent dye and the second fluorescent dye to determine the number of living microorganisms and that of dead microorganisms.

While the exemplary embodiments have been shown and described, it will be understood by those skilled in the art that various changes in form and details may be made thereto without departing from the spirit and scope of the present disclosure as defined by the appended claims.

In addition, many modifications can be made to adapt a particular situation or material to the teachings of the present disclosure without departing from the essential scope thereof. Therefore, it is intended that the present disclosure not be limited to the particular exemplary embodiments disclosed as the best mode contemplated for carrying out the present disclosure, but that the present disclosure will include all embodiments falling within the scope of the appended claims.

What is claimed is:

1. An apparatus for evaluating efficiency of an antimicrobial air filter, comprising:
a microorganism-supplying device supplying a microorganism-containing solution by spraying the microorganism-containing solution in a gas phase;
a drying device drying the microorganisms-containing solution sprayed in a gas phase to dry a solvent in the solution;
an antimicrobial air filter chamber provided with an antimicrobial air filter and providing a space in which the microorganisms supplied from the drying device are attached to the antimicrobial air filter;
a fluorescent dyeing device for the antimicrobial air filter carrying out fluorescent dyeing of the antimicrobial air filter by using a fluorescent dye containing a combination of a first fluorescent dye capable of fluorescent dyeing of living microorganisms and a second fluorescent dye capable of fluorescent dyeing of dead microorganisms; and
a fluorescence analysis device measuring the fluorescence emitted from the antimicrobial air filter fluorescent-dyed by the fluorescent dyeing device to perform quantitative analysis of the living microorganisms and dead microorganisms.

2. The apparatus according to claim 1, wherein the first fluorescent dye and the second fluorescent dye emit fluorescence which differs from each other in wavelength.

3. The apparatus according to claim 1, wherein the first fluorescent dye is Syto9 and the second fluorescent dye is propidium iodide.

4. The apparatus according to claim 1, wherein the microorganism-containing solution sprayed in a gas phase and the microorganisms in the drying device are transferred by compressed air, and the compressed air is generated by an air compressor.

5. The apparatus according to claim 1, wherein the fluorescent dyeing device for the antimicrobial air filter comprises a fluorescent dye supplying device and a substrate, the antimicrobial air filter having the microorganisms attached thereto are mounted to the substrate, and the fluorescent dyes are sprayed onto the antimicrobial air filter through the fluorescent dye supplying device, so that the antimicrobial air filter is dyed with the fluorescent dye.

6. The apparatus according to claim 1, wherein the fluorescence analysis device comprises a fluorescence filter block and a fluorescence microscope, the fluorescence filter block serves to filter specific fluorescence emitted from each of the fluorescent-dyed living microorganisms and dead microorganisms, and the fluorescence microscope recognizes shapes of microorganisms and the fluorescence emitted from microorganisms to determine number of the living microorganisms and count of the dead microorganisms.

7. A method for evaluating the efficiency of an antimicrobial air filter, comprising:
spraying a microorganism-containing solution in a gas phase;
drying the microorganism-containing solution sprayed in a gas phase;
attaching microorganisms to an antimicrobial air filter; and
carrying out fluorescent dyeing of the antimicrobial air filter by using a fluorescence dye containing a combination of a first fluorescent dye capable of fluorescent dyeing of living microorganisms and a second fluorescent dye capable of fluorescent dyeing of dead microorganisms; and
measuring the fluorescence emitted from the antimicrobial air filter dyed with the first fluorescent dye and the second fluorescent dye to determine number of living microorganisms and that of dead microorganisms.

8. The method according to claim 7, wherein the first fluorescent dye and the second fluorescent dye emit fluorescence which differs from each other in wavelength.

9. The apparatus according to claim 7, wherein the first fluorescent dye is Syto9 and the second fluorescent dye is propidium iodide.

10. The method according to claim 7, wherein the microorganism-containing solution sprayed in a gas phase and the microorganisms are transferred by compressed air.
